(19) United States
(12) Patent Application Publication
(10) Pub. No.: US 2016/0271371 A1
(43) Pub. Date: Sep. 22, 2016

(54) GUIDEWIRE SYSTEM AND METHOD FOR ADVANCEMENT THROUGH A TORTUROUS PATH

(71) Applicant: Cardica, Inc., Redwood City, CA (US)

(72) Inventors: Bernard A. Hausen, Redwood City, CA (US); Brendan M. Donohoe, Fairfax, CA (US)

(21) Appl. No.: 14/817,049

(22) Filed: Aug. 3, 2015

Related U.S. Application Data
(60) Provisional application No. 62/136,039, filed on Mar. 20, 2015.

(51) Int. Cl. A61M 25/09; A61M 25/00

(52) U.S. Cl. CPC A61M 25/09; A61M 25/0026; A61B 2017/00292

(57) ABSTRACT

A surgical guiding device for minimally invasive surgical procedures includes a flexible elongated body having one or more through lumens; and a guidewire system at least partially disposed within at least one of said one or more through lumens of the flexible elongated body for guiding a surgical instrument to a surgical site.

### FIELD OF THE INVENTION

The present invention relates generally to surgical devices; and more particularly, to guidewire systems and methods to advance or deliver a surgical instrument through a tortuous path to a target surgical site.

### BACKGROUND

Minimally invasive surgical procedures are becoming more common as surgeons use a variety of techniques to operate with reduced trauma to the body than with open surgeries. In general, minimally invasive surgical procedures are safer than open surgeries and allow the patient to recover faster and heal with less pain and scarring. Minimally invasive surgical procedures are typical performed with one or more incisions to the body where surgical instruments are inserted and maneuvered into the surgical site to treat the targeted tissue.

Accordingly, it would be desirable to develop systems and methods to facilitate minimally invasive surgical procedures.

### SUMMARY OF THE INVENTION

A surgical guiding device for minimally invasive surgical procedures includes a flexible elongated body having one or more through lumens, and a guidewire system at least partially disposed within at least one of said one or more through lumens of the flexible elongated body for guiding a surgical instrument to a surgical site.

A surgical guiding device for minimally invasive surgical procedures includes a flexible elongated body having one or more through lumens, and a guidewire system at least partially disposed within at least one of said one or more through lumens of the flexible elongated body for guiding a surgical instrument to a surgical site. The guidewire system includes a guidewire member. The guidewire member is a looped-belt, a looped-cable, or a similar looped-element.

The guidewire system of the surgical guiding device includes a guiding member for guiding the movement of the guidewire member. The guide member prevents the guidewire member from binding or kinking. Also, the guidewire system includes one or more guide track members to maintain at least one portion of the guidewire member in a loop.

The guidewire system as disclosed includes a carrier member which is configured to guide a surgical instrument from a proximal portion of the surgical guiding device to a distal portion of the surgical guiding device. The surgical instrument may be removably couple to the carrier member.

The surgical guiding device may further include a positioning member which is configured to position or move a surgical instrument that is coupled to the guidewire system from a first position to a second position. For example, the first position may be substantially near the elongated body of the surgical guiding device, while the second position may be substantially near a target tissue. In other words, the positioning member can position or move a surgical instrument to or near a target tissue for performing various surgical procedures or treatments. The positioning member may be a substantial long and/or slender or narrow strip of material. In operation, the positioning member may be deflected from a first configuration to a second configuration. The positioning member may be substantially flexible.

### BRIEF DESCRIPTION OF THE DRAWINGS

The present invention will be readily understood by the following disclosure, taken in conjunction with accompanying drawings, illustrating by way of examples of the embodiments of the invention. The figures are merely exemplary and not limiting. The objects and elements in the drawings are not necessarily drawn to scale, proportion, precise orientation or positional relationships; instead, emphasis is focused on illustrating the principles of the invention. Descriptive terms such as “upper,” “lower,” “upward,” “downward”, “front”, “back”, “forward”, “backward”, and the like are intended for the convenience of the reader and refer to the orientation and/or motion of parts as illustrated and described; they do not necessarily limit the orientation or operation of the features, aspects, or embodiments of the invention. The drawings illustrate the design and utility of various features, aspects, or embodiments of the present invention. The drawings, however, depict the features, aspects, or embodiments of the invention, and should not be taken as limiting in their scope. With this understanding, the features, aspects, or embodiments of the invention will be illustrated through the use of the accompanying drawings in which:

FIG. 1 illustrates an example of a surgical guiding instrument having one or more lumens or channels in a substantial elongate and flexible body, in accordance with features, aspects or embodiments of the present invention.

FIG. 2 illustrates a cross-sectional view of the surgical guiding instrument/colonoscopy having one or more through lumens or channels, in accordance with features, aspects or embodiments of the present invention.

FIG. 3 illustrates a top cross-sectional view of the surgical guiding instrument with a guidewire system incorporated into at least one lumen or channel of the surgical instrument, in accordance with features, aspects or embodiments of the present invention.

FIG. 4 illustrates an isometric cross-sectional view of the surgical guiding instrument with a guidewire system incorporated into at least one lumen or channel of the surgical instrument, in accordance with features, aspects or embodiments of the present invention.

FIG. 5 illustrates a frontal isometric view of a guidewire system that is configured to be coupled to or adopted with a surgical guiding instrument having at least one through lumen or channel for guiding surgical operating instruments from a proximal portion of the surgical instrument to a distal portion of the surgical instrument, in accordance with features, aspects or embodiments of the present invention.

FIG. 6 illustrates a back isometric view of a guidewire system that is configured to be coupled to or adopted with a surgical guiding instrument having at least one through lumen or channel for guiding surgical operating instruments from a proximal portion of the surgical instrument to a distal portion of the surgical instrument, in accordance with features, aspects or embodiments of the present invention.

FIG. 7 illustrates a backward isometric view of a guidewire system that is configured to be coupled to or adopted with a surgical guiding instrument having at least one through lumen or channel for guiding surgical operating instruments from a proximal portion of the surgical instrument to a distal portion of the surgical instrument, in accordance with features, aspects or embodiments of the present invention.

FIG. 8A illustrates a guidewire conveyor system with a carrier feature for guiding a surgical operating instrument to a desired surgical site, in accordance with features, aspects or embodiments of the present invention.

FIG. 8B illustrates a guidewire conveyor system with a carrier feature for guiding a surgical operating instrument to a desired surgical site, in accordance with features, aspects or embodiments of the present invention. For example, a surgical operating instrument may be removably coupled to the carrier feature and allow the carrier feature to guide the surgical operating instrument to the desired surgical site.

FIG. 9A illustrates a top view of a guidewire conveyor system with a carrier feature for guiding a surgical operating instrument to a desired surgical site, in accordance with features, aspects or embodiments of the present invention. For example, a surgical operating instrument may be removably coupled to the carrier feature and allow the carrier feature to guide the surgical operating instrument to the desired surgical site. As illustrated, the surgical operating instrument may be guided from a first position.

FIG. 9B illustrates a top view of a guidewire conveyor system with a carrier feature for guiding a surgical operating instrument to a desired surgical site, in accordance with features, aspects or embodiments of the present invention.

FIG. 10A illustrates an isometric view of a guidewire conveyor system with a carrier feature for guiding a surgical operating instrument to a desired surgical site, in accordance with features, aspects or embodiments of the present invention.

FIG. 10B illustrates a top view of a guidewire conveyor system with a carrier feature for guiding a surgical operating instrument to a desired surgical site, in accordance with features, aspects or embodiments of the present invention.

FIG. 11 illustrates that the guidewire system (coupled to a surgical instrument/colonoscopy) may be retracted or withdrawn away from a surgical operating instrument after the surgical operating instrument has been guided (e.g., through a tortuous path) to a surgical site, in accordance with features, aspects or embodiments of the present invention.

FIG. 12 illustrates a frontal view of a guidewire system (coupled to a surgical instrument/colonoscopy) with a surgical operating instrument inside a cavity or a surgical site, in accordance with features, aspects or embodiments of the present invention.

FIG. 13 illustrates a frontal view of a guidewire system (coupled to a surgical instrument/colonoscopy) with a surgical operating instrument inside a cavity or a surgical site, wherein the surgical operating instrument may include a positioning feature to allow the surgical operating instrument to be maneuver over a target tissue, in accordance with features, aspects or embodiments of the present invention.

FIG. 14A illustrates a positioning feature/element of a surgical operating instrument in an un-deployed state, in accordance with features, aspects or embodiments of the present invention.

FIG. 14B illustrates a positioning feature/element of a surgical operating instrument in a deployed state, in accordance with features, aspects or embodiments of the present invention.

### DETAILED DESCRIPTION

In the following detailed description, specific details are set forth in order to provide a thorough understanding of the present invention. However, it will be readily understood by those skilled in the art that the present invention may be practiced without these specific details. Alternatively, some of the well-known parts, components, hardware, methods of operations, and procedures may not be described in detail or elaborated so as to avoid obscuring the present invention; but, nevertheless, they are within the spirit and scope of the present invention.

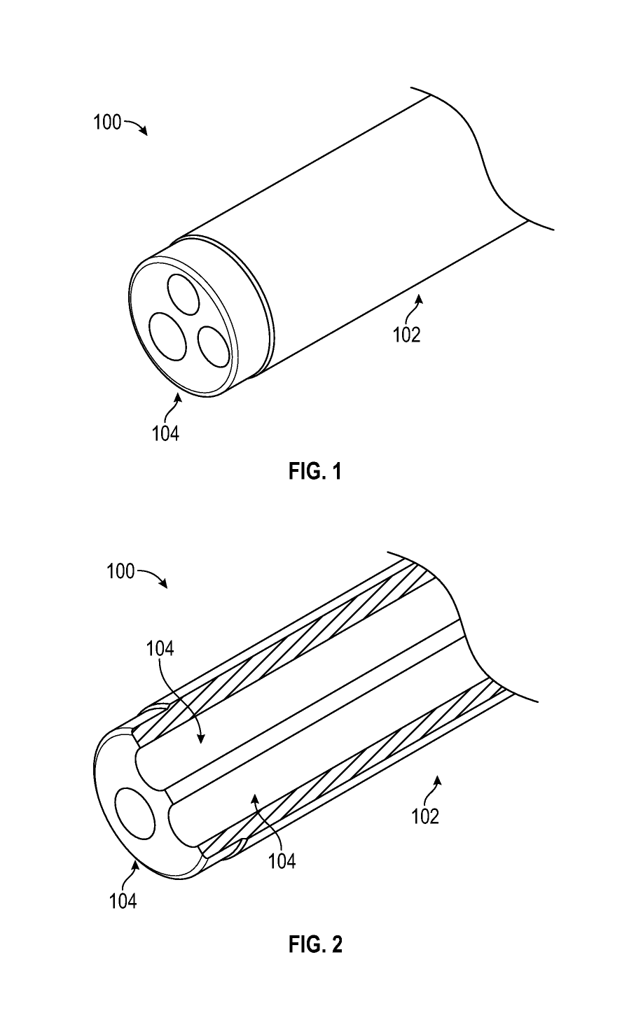

FIG. 1 illustrates an example of a surgical guiding instrument 100, in accordance with embodiments of the present invention. The surgical guiding instrument 100 may be constructed and/or configured for exploring surgical sites and to locate target tissues for treatment in minimally invasive surgical procedures. For example, the surgical guiding instrument 100 may have a substantially flexible and elongated body 102 that is suited for navigating through tortuous body cavities or pathways inside a patient. In some embodiments, the surgical guiding instrument 100 may be articulated at various regions, segments or control points along the elongated body 102 of the guiding instrument 100. In other words, the surgical guiding instrument 100 may be articulated, navigated, advanced and/or retreated in the body cavities or pathways inside a patient. In some embodiments, the surgical guiding instrument 100 may not be used through tortuous cavities or pathways; as such the elongated body 102 may alternatively be substantially rigid. The surgical guiding instrument 100 may be configured to operate as a colonoscope, in addition to being operated or used as a guiding or guidewire-like system. The surgical guiding instrument 100 may have one or more lumens 104 or channels 104 that could serve as pass-throughs or tunnels that originate from a proximal portion of the guiding instrument 100 to a distal portion of the guiding instrument 100. In some embodiments, the guiding instrument 100 may be configured or equipped with optics of various kinds, e.g., fiber optics, infrared optics, etc., such that the guiding instrument 100 can be used as an optical instrument, such as a colonoscope, in addition to being a guiding instrument. In such embodiments, the optics or optical instrument(s) may be disposed within one or more of the lumens 104 in the elongated body 102 of the surgical guiding instrument 100. In other words, for some embodiments, the surgical instrument may be operated as a colonoscope with one or more lumens or channels and an optical device. Surgical operating instruments may be passed through the one or more lumens or channels from the proximal portion of the colonoscope to the distal portion of the colonoscope. The optical device may provide various visual images to the operating surgeon to view the surgical site at the distal portion of the colonoscope.

FIG. 2 illustrates a cross-sectional view of the surgical guiding instrument 100 having one or more through lumens 104 or channels 104, in accordance with features, aspects or embodiments of the present invention. For example, the one or more through lumens 104 or channel 104 may allow various surgical operating instruments to be passed through from the proximal portion of the guiding instrument 100 to the distal portion of the guiding instrument 100. As may be appreciated, a surgeon may initially use the guiding instrument 100, with optical capabilities, to explore a surgical site and find a targeted tissue for treatment. Once the targeted tissue is located, various surgical instrument(s) can be passed through one or more of the lumens 100 of the guiding instrument 100. That is, without removing the guiding instrument, the tip or distal portion of the guiding instrument 100 can be used as the point-of-treatment for a surgical procedure. The delivered surgical instrument(s) can then be used to perform various surgical treatments on the target tissue, and once the treatment is completed, the surgical instrument(s) can then be retrieved back through the lumens 104 of the guiding instrument 100.

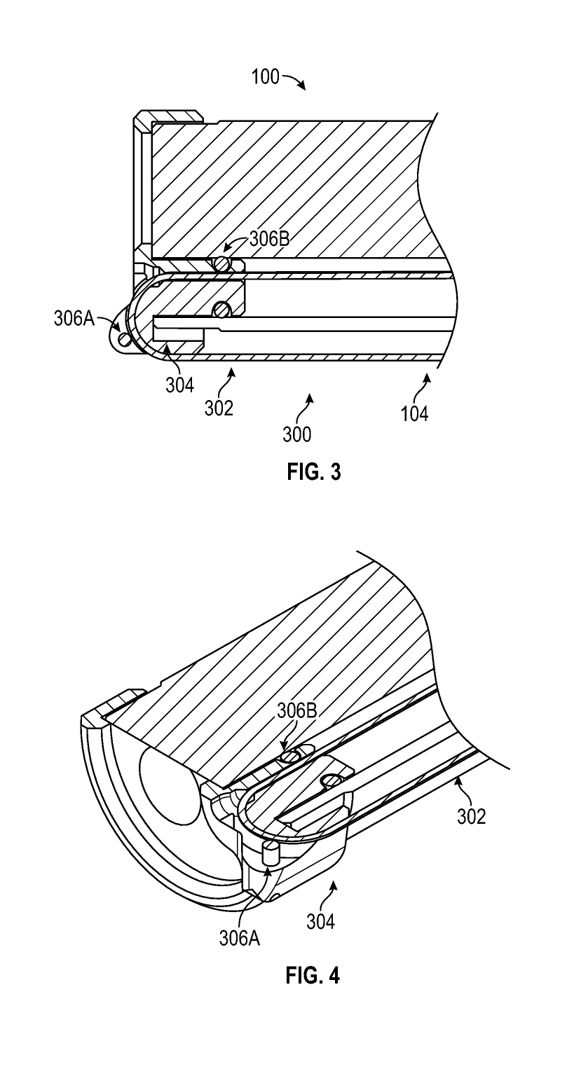

FIG. 3 illustrates a top cross-sectional view of the surgical guiding instrument 100 with a guidewire system 300 incorporated into at least one lumen 104 or channel 104 of the surgical guiding instrument 100, in accordance with features, aspects or embodiments of the present invention. For example, the guidewire system 300 may be configured in a conveyor-like manner, in which a substantially flexible guidewire 302 may be wrapped around a substantially rounded guide member 304 to allow the guidewire 302 to move about in a conveyor-belt like manner. Also as illustrated in FIG. 3, the guide member 304 may be configured to substantially constraint the guidewire 302 and to position the guidewire 302 around the guide member 304. It may also allow the guidewire 302 move substantially freely in a loop-fashion around the guide member 304 without binding or kinking. Furthermore, additional guide elements such as a guide track member 306A and guide track member 306B may be properly positioned, guided and secured the guidewire 302 to ensure that the path or path of travel of the guidewire 302 is proper constrained. As illustrated, this guidewire system 300 may be advantageously installed or positioned near the distal portion of the guiding instrument 100. As will be discussed in further details, the guidewire system 300 may be used to deliver various surgical instrument(s) to the distal portion of the guiding instrument 100. As may be appreciated, the distal portion of the guiding instrument 100 may be substantially the “working” end of the instrument where treatment of targeted tissue will be carried out at about or in the proximity of the distal tip of the guiding instrument 100. Hence, the guiding system 100 may be configured to be used substantially as a guidewire to locate a target tissue for surgical procedures. For example, using the lumens 104 with optical instrument disposed therein to find a targeted tissue or operational site, the guiding instrument 100 provides optical capabilities to a surgical to “see”, find, and locate a surgical site. In addition, one of more lumens 104 may be used to deliver surgical instruments within the instrument body 102 of the guiding instrument 100. Alternatively, the guidewire system 300 may be used to deliver surgical instruments externally of the instrument body 102 of the guiding instrument 100.

FIG. 4 illustrates an isometric cross-sectional view of the surgical instrument 100 with a guidewire system 300 incorporated into at least one lumen 104 or channel 104 of the surgical instrument 100, in accordance with features, aspects or embodiments of the present invention. The isometric cross-sectional view provides another perspective of the surgical guiding instrument. As illustrated, for example, the guidewire system 300 may be configured in a conveyor-like manner, in which a substantially flexible guidewire 302 may be wrapped around a substantially rounded guide element 304 to allow the guidewire 302 to move about in a conveyor-belt like manner. Also as illustrated in FIG. 4, the guide element 304 may include various members 306A and 306B to substantially constraint the guidewire 302 to position the guidewire around the guide element 304.

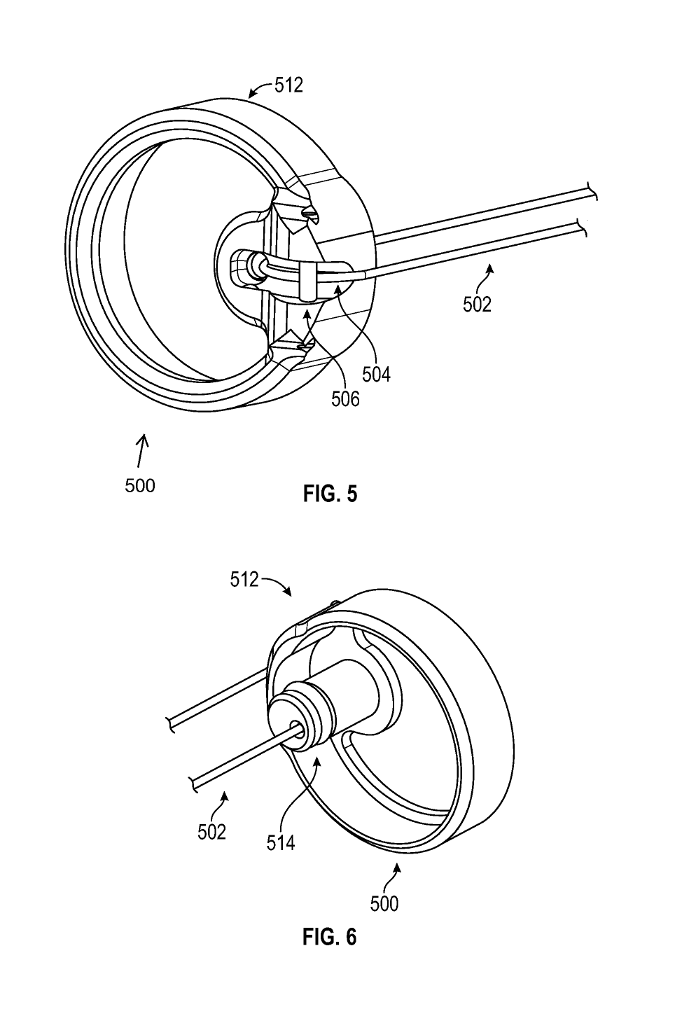

FIG. 5 illustrates a frontal isometric view of a guidewire system that is configured to be coupled to or adopted with a surgical instrument having at least one through lumen or channel for guiding surgical operating instruments from a proximal portion of the surgical instrument to a distal portion of the surgical instrument, in accordance with features, aspects or embodiments of the present invention. As illustrated, a guidewire collar system 500 is configured to be coupled, incorporated or adopted to one end (e.g., distal end) of an instrument (e.g., a guiding instrument) to guiding or tracking capabilities. For example, guidewire collar system 500 may include a guidewire collar member 512 configured to couple or fitted to an operational end of an instrument, such as the guiding instrument 100. The guidewire system 500 may also include a substantially rounded guide member 504 to guide a guidewire member 502. The guidewire member 502 may be moved, advanced or retreated in a conveyor-belt like manner around the guide member 504. In addition, the guidewire system 500 may also include one or more constraint members 506 to further secure the guidewire member 502 in its position and movement about the guide member 504.

FIG. 6 illustrates a rear or backward isometric view of the guidewire system 500 that is configured to be coupled to or adopted with a surgical instrument (e.g., 100) having at least one through lumen or channel for guiding surgical operating instruments from a proximal portion of the surgical instrument to a distal portion of the surgical instrument, in accordance with features, aspects or embodiments of the present invention. In addition to the features, elements, and members as described in FIG. 5, the guidewire system 500 may also include a guidewire support member 514 to provide further support to the guidewire 502, particular to the interior portion of the guidewire 502 while it is being operated, e.g., moved, advanced or retracted.

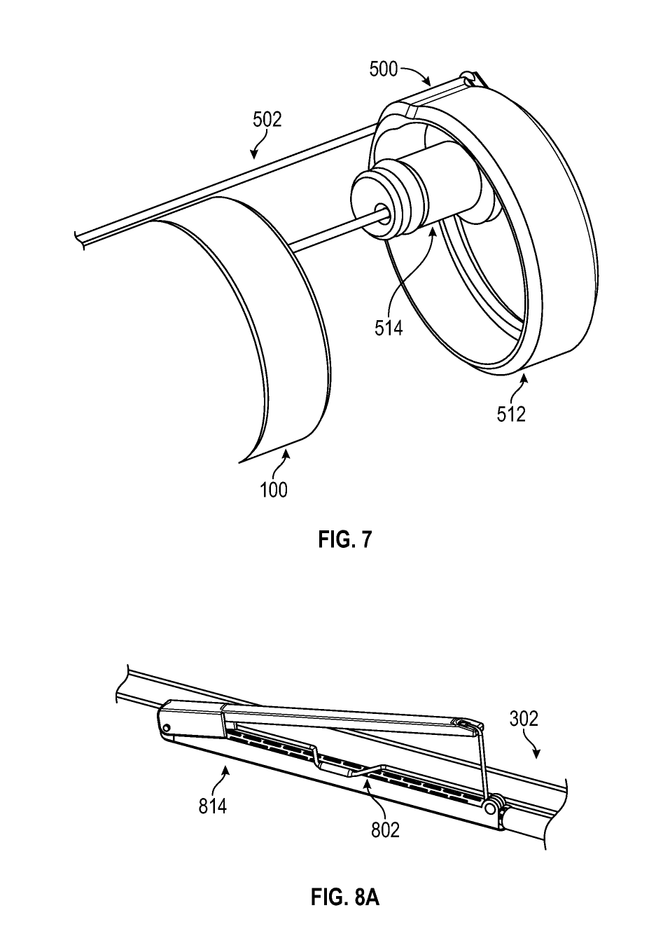

FIG. 7 illustrates a rear or backward isometric view of the guidewire system 500 that is configured to be coupled to or adopted with a surgical instrument (e.g., surgical instrument 100) having at least one through lumen or channel for guiding surgical operating instruments from a proximal portion of the surgical instrument to a distal portion of the surgical instrument, in accordance with features, aspects or embodiments of the present invention. As illustrated, the guidewire system 500 may be coupled, adopted or fitted to one end (e.g., the distal end, the operational end, etc.) of a surgical instrument (such as the guiding instrument 100).

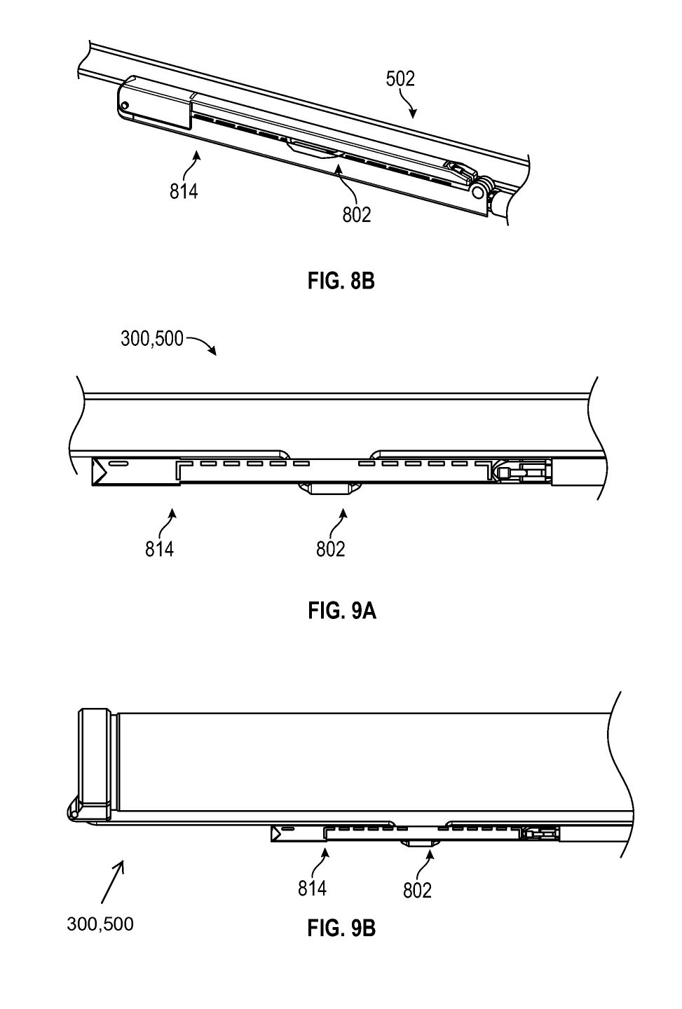

FIG. 8A illustrates a guidewire conveyor system (300, 500) with a carrier feature 802 incorporated into a guidewire (302, 502) for guiding a surgical operating instrument 814 to a desired surgical site, in accordance with features, aspects or embodiments of the present invention. In some embodiments, the surgical operating instrument may couple directly onto the guidewire (302, 502) with the carrier feature 802. FIG. 8B illustrates the guidewire conveyor system with a carrier feature for guiding a surgical operating instrument to a desired surgical site, in accordance with features, aspects or embodiments of the present invention. For example, a surgical operating instrument 814 may be removably coupled to the carrier feature 802 and allow the carrier feature 802 to guide the surgical operating instrument to the desired surgical site. The surgical operating instrument 814 may clamp on or otherwise grasp onto the carrier feature 802 or directly onto the guidewire (302, 502).

FIG. 9A illustrates a top view of a guidewire conveyor system (300, 500) with a carrier feature 802 for guiding a surgical operating instrument 814 to a desired surgical site, in accordance with features, aspects or embodiments of the present invention. For example, a surgical operating instrument 814 may be removably coupled to the carrier feature 802 and allow the carrier feature 802 to guide the surgical operating instrument 814 to the desired surgical site. As illustrated, the surgical operating instrument 814 may be guided from a first position. FIG. 9B illustrates a top view of a guidewire conveyor system (300, 500) with a carrier feature 802 for guiding a surgical operating instrument 814 to a desired surgical site, in accordance with features, aspects or embodiments of the present invention. For example, a surgical operating instrument 814 may be removably coupled to the carrier feature 802 and allow the carrier feature 802 to guide the surgical operating instrument 814 to the desired surgical site. As illustrated, the surgical operating instrument 814 may be guided to a second position. Alternatively, the surgical operating instrument 814 may be guided to a distal portion of the guidewire conveyor system (300, 500).

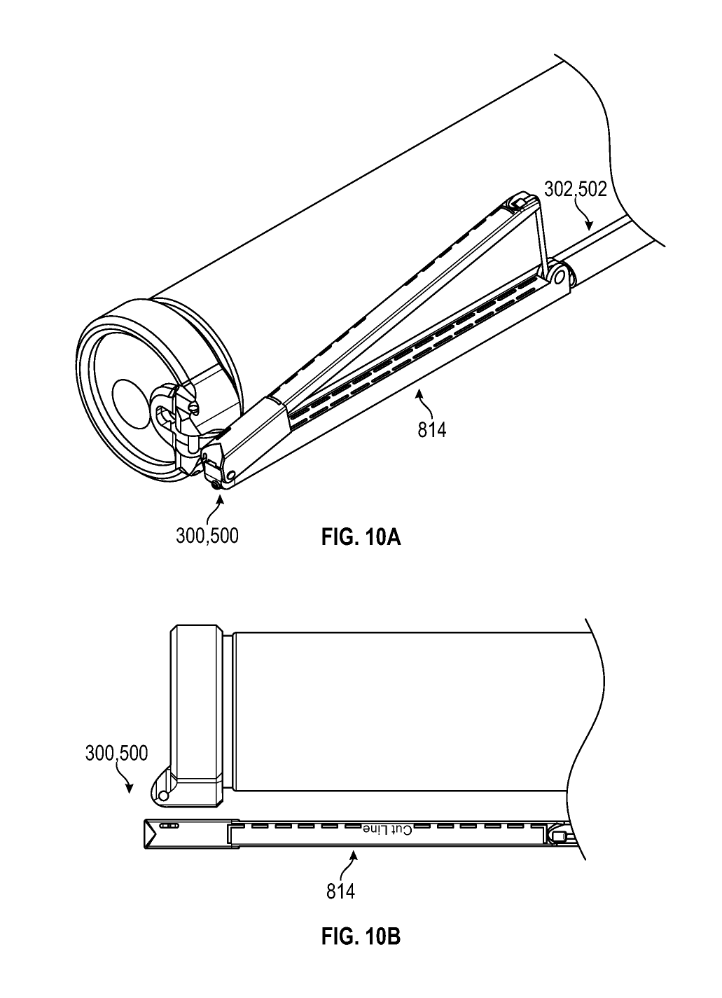

FIG. 10A illustrates an isometric view of a guidewire conveyor system (300, 500) with a carrier feature 802 for guiding a surgical operating instrument 814 to a desired surgical site, in accordance with features, aspects or embodiments of the present invention. For example, a surgical operating instrument 814 may be removably coupled to the carrier feature 802 and allow the carrier feature 802 to guide the surgical operating instrument 814 to the desired surgical site. As illustrated, the surgical operating instrument may be guided to a distal portion of the guidewire conveyor system (300, 500). FIG. 10B illustrates a top view of a guidewire conveyor system (300, 500) with a carrier feature 802 for guiding a surgical operating instrument 814 to a desired surgical site, in accordance with features, aspects or embodiments of the present invention. For example, a surgical operating instrument 814 may be removably coupled to the carrier feature 802 and allow the carrier feature 802 to guide the surgical operating instrument 814 to the desired surgical site. As illustrated, the surgical operating instrument 814 may be guided to a distal portion of the guidewire conveyor system (300, 500).

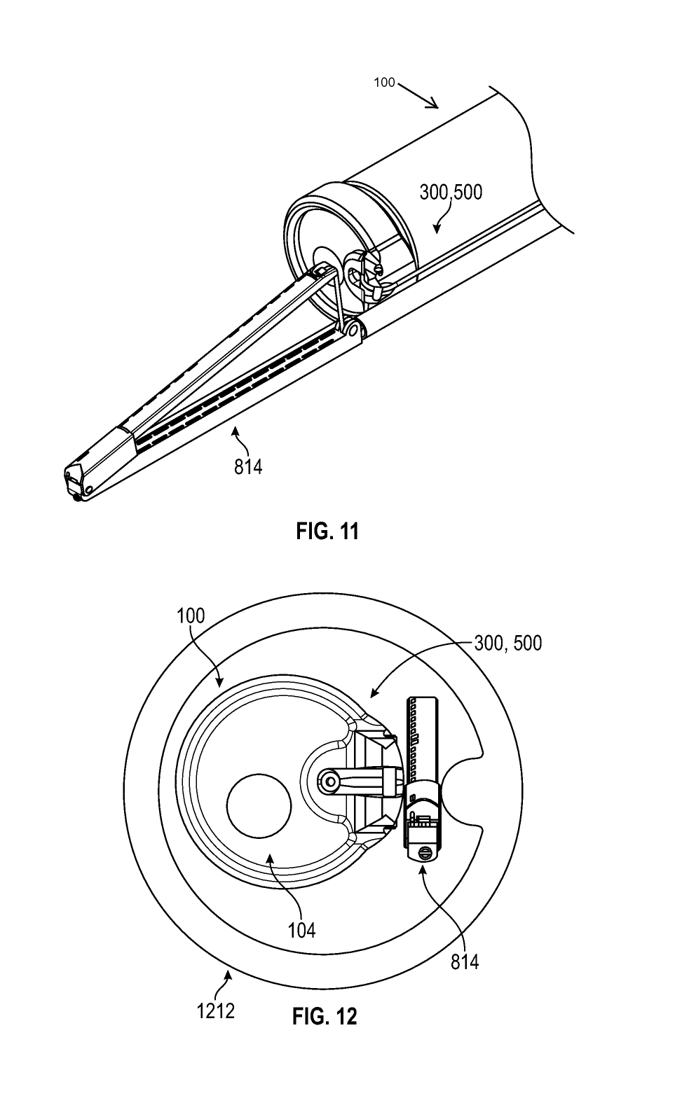

FIG. 11 illustrates that the guidewire system 300, 500 (coupled to a surgical instrument 100/colonoscope) may be used to guide a surgical instrument 814 (e.g., through a tortuous path) to a surgical site, and then the guidewire system 300, 500 (coupled to surgical guiding instrument 100) may be retracted or withdrawn away from the surgical operating instrument 814 leaving it at surgical site for performing various surgical procedures, in accordance with features, aspects or embodiments of the present invention. Alternatively, the guidewire system 300, 500 (coupled to a surgical instrument 100/colonoscope) may be used to retrieve or recover the surgical instrument 814 away from a surgical operating site after it has completed the surgical procedures at the surgical site.

FIG. 12 illustrates a frontal view of a guidewire system 300, 500 (coupled to a surgical instrument 100/colonoscope) with a surgical operating instrument 814 inside a cavity 1212 of a surgical site, in accordance with features, aspects or embodiments of the present invention. For example, the surgical site may be a colon and the surgical operating instrument may be a cutter configured to remove/cut a colon polyp.

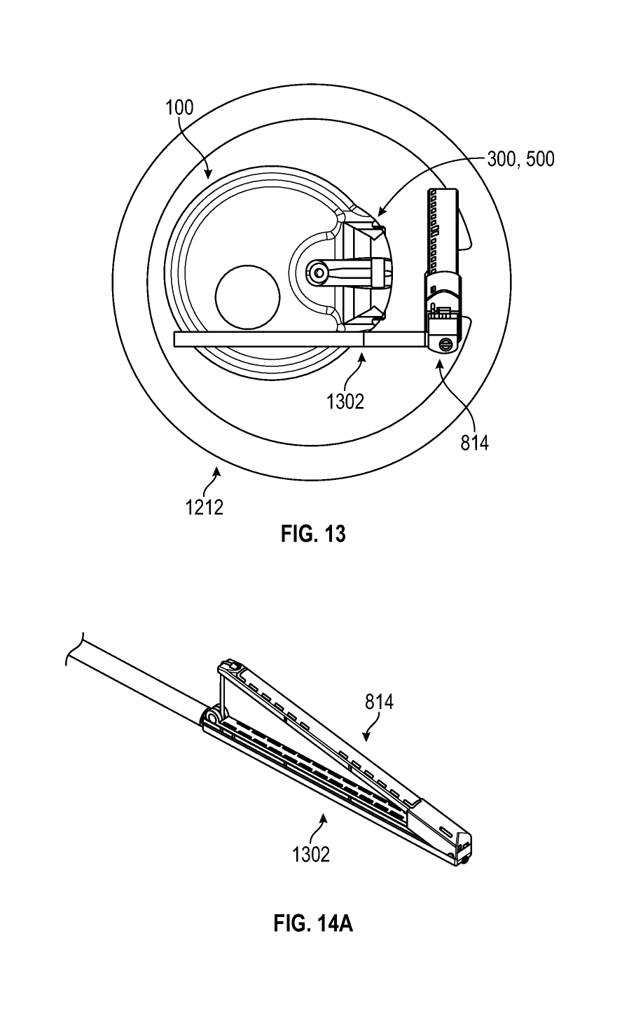

FIG. 13 illustrates a frontal view of a guidewire system 300, 500 (coupled to a surgical instrument 100/colonoscopy) with a surgical operating instrument 814 inside a cavity 1212 of a surgical site, wherein the surgical operating instrument may include a positioning member 1302 to allow the surgical operating instrument 814 to be maneuver over a target tissue, in accordance with features, aspects or embodiments of the present invention. For example, the positioning member 1302 may allow the surgical operating instrument/cutter to be maneuvered over a polyp for cutting or removal. Alternatively, the positioning member 1302 may be incorporated or coupled to the elongate body 102 of the guiding instrument 100.

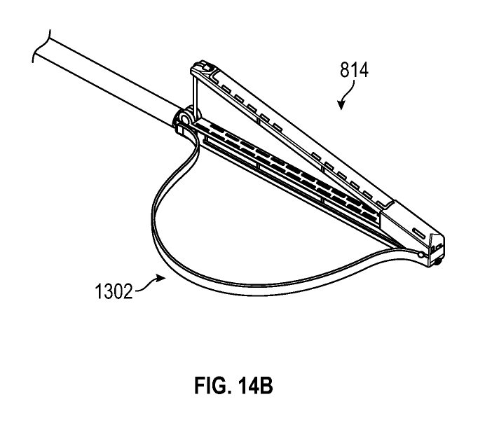

FIG. 14A illustrates a positioning member/element 1302 of a surgical operating instrument 814 in an un-deployed state, in accordance with features, aspects or embodiments of the present invention. Alternatively, the positioning member/element 1302 may be incorporated or coupled to the elongate body 102 of the guiding instrument 100. FIG. 14B illustrates the positioning feature/element 1302 of the surgical operating instrument 814 in a deployed state, in accordance with features, aspects or embodiments of the present invention. For example, the positioning feature/element 1302 may be a substantially stiff but flexible element that can be made to flex or deflect into various configurations (by a control member—by pushing or pulling onto the positioning member). Furthermore, in a deployed state, the positioning feature/element 1302 may “push” or “move” the surgical operating instrument 814 into a desired location or position for deployment upon a target tissue.

Multiple features, aspects, and embodiments of the invention have been disclosed and described by the illustrated figures. Many combinations and permutations of the disclosed invention may be useful in operating a surgical stapling device, and the invention may be configured to support various surgical procedures. One of ordinary skill in the art having the benefit of this disclosure would appreciate that the foregoing illustrated and described features, aspects, and embodiments of the invention may be modified or altered, and it should be understood that the invention generally, as well as the specific features, aspects, and embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure.

Although particular features, aspects, and embodiments of the present invention have been illustrated and described, it should be understood that the above disclosure is not intended to limit the present invention to these features, aspects, and embodiments. It will be obvious to those skilled in the art that various changes and modifications may be made without departing from the spirit and scope of the present invention. Thus, the present invention is intended to cover alternatives, modifications, and equivalents that may fall within the spirit and scope of the following claims and their equivalents.

What is claimed:

1. A surgical guiding device for minimally invasive surgical procedures, comprising:
a flexible elongated body having one or more through lumens; and
a guidewire system at least partially disposed within at least one of said one or more through lumens of the flexible elongated body.

2. The surgical guiding device of claim 1, wherein the guidewire system includes a guidewire member.

3. The surgical guiding device of claim 2, wherein the guidewire member is a looped-belt or a looped-cable.

4. The surgical guiding device of claim 2, wherein the guidewire system includes a guide member for guiding the movement of the guidewire member.

5. The surgical guiding device of claim 4, wherein the guide member prevents the guidewire member from binding or kinking.

6. The surgical guiding device of claim 4, wherein the guidewire system includes one or more guide track members to maintain at least one portion of the guidewire member in a loop.

7. The surgical guiding device of claim 1, wherein the guidewire system includes a guidewire collar member, the guidewire collar member allows the guidewire system to be removably coupled to the elongated body of the surgical guiding device.

8. The surgical guiding device of claim 2, wherein the guidewire member includes a carrier member, wherein the carrier member guides a surgical instrument from a proximal portion of the surgical guiding device to a distal portion of the surgical guiding device.

9. The surgical guiding device of claim 1 further comprising a positioning member, the positioning member is configured to position or move a surgical instrument coupled to the guidewire system from a first position to a second position.

10. The surgical guiding device of claim 9, wherein the first position is substantially near the elongated body of the surgical guiding device while the second position is substantially near a target tissue.

11. The surgical guiding device of claim 9, wherein the positioning member is a long, slender strip.

12. The surgical guiding device of claim 9, wherein the positioning member is deflected from a first configuration to a second configuration.

13. The surgical guiding device of claim 12, wherein the positioning member is substantially flexible.
